(19) United States
(12) Patent Application Publication
(10) Pub. No.: US 2010/0298934 A1
(43) Pub. Date: Nov. 25, 2010

(54) BREAST PROSTHESIS

(75) Inventor: Nils Stelter, Frasdorf (DE)

Correspondence Address: DILWORTH & BARRESE, LLP, 1000 WOODBURY ROAD, SUITE 405, WOODBURY, NY, 11797, US

(73) Assignee: Amoena Medizin Orthopaedie-Technik GmbH, Raubling (DE)

(21) Appl. No.: 12/782,088

(22) Filed: May 18, 2010

(30) Foreign Application Priority Data
May 18, 2009 (DE) UM202009007115.4

(51) Int. Cl. A61F 2/12

(52) U.S. Cl. 623/7

(57) ABSTRACT

The present invention relates to a removable, external breast prosthesis, comprising a body substantially corresponding to the shape and firmness of the female breast and made from one or more flexible plastic parts, wherein the side of the prosthesis facing the skin has antimicrobial properties due to an antimicrobial coating and/or to antimicrobial material in the matrix.

The present invention relates to a removable, external breast prosthesis comprising a body which substantially corresponds to the shape and firmness of the female breast, which comprises one or more flexible plastic parts and which has antimicrobial properties on the side facing the skin.

Removable, external breast prostheses were developed for women whose breast had to be amputated in full or in part in the course of a mastectomy due to breast cancer or to another disease of the breast. The aim of both postoperative and permanent wearing of a breast prosthesis is primarily to increase the self-confidence of an affected woman after ablatio mammae. To keep the restrictions in lifestyle as small as possible, high demands on such prostheses are made with respect to the visual appearance and to haptic perception so that the shape and firmness of the natural breast is reproduced as much as possible. Low weight, or a weight comparable with the natural breast, high comfort in wear, good adhesion to the skin, low heat build-up and good compatibility and hygiene between the prosthesis and the affected area of skin, frequently sensitized by cicatrization are equally important to guarantee comfortable, slip-free and secure wearing over a longer time period, also while moving.

Due to these high requirements and to the high demand (breast cancer is the most frequent form of cancer in women in the western world), a lot of research takes place in this area and many different breast prostheses are consequently known from the prior art.

To secure a prosthesis in as stable a manner as possible to the skin of the wearer, it is known from EP 0 392 960 A1 to provide breast prostheses within a peripheral lip-shaped margin on their rear side with a likewise peripheral shoulder formed by a ledge. Adhesive strips or adhesive parts are secured to this shoulder which, while cooperating with the adhesive regions of the strips adhered to the body of the woman, releasably connect the prosthesis to the skin. A hook-and-loop type connection is provided as the securing means in this respect, for example.

DE 27 01 627 A1 discloses breast prostheses of bodies welded into plastic films, emulating the shape of the breast and made from an addition cross-linking two-component silicone rubber compound. Such prostheses reproduce the properties of the natural breast very well due to their elastic softness, the reaction of the material to movement, the tangible consistence and the weight of the material used. DE 90 10 426 U1 also proposes breast prostheses made of a shell-shaped body of a flexibly set, addition cross-linking two-component silicone rubber which is welded in plastic films.

To increase the comfort in wear, a breast prosthesis was proposed in DE 201 01 174 U1 which is likewise made of bodies welded in plastic films, emulating the shape of the breast and comprising an addition cross-linking two-component silicone rubber compound. However, at least on their side facing the body, these in part have a PCM (phase conversion material) material which has a phase conversion temperature in the region of the body temperature. This PCM material can also be located in a separate pocket facing the body. The comfort in wear is hereby substantially increased by avoiding the heat build-up between the prosthesis and the skin of the wearer. A design of a two-chamber breast prosthesis likewise counteracting a heat build-up between the skin of the wearer and the prosthesis is disclosed by DE 44 21 516 A1. A cushion body of foam plastic or a fiber clew is present on the side facing the skin here.

EP 0 768 068 B1 shows a further two-chamber breast prosthesis. The side facing the skin here has a tixotropic, preferably pasty material which is made, for example, from a mixture of non cross-linked silicone oil and silicic acid. Such a prosthesis is in particular suitable for patients with a partially amputated breast or an uneven scar development due to its particularly good adaptation capability. EP 0 778 013 A1 combats the same problem. Here, the filler material of the second chamber facing the skin is a cross-linking material which adapts to irregularities when placed on and substantially maintains this shape.

A very large variety of shapes and sizes can be achieved by breast prostheses in accordance with the German utility model 20 2008 008 907-7. Such a prosthesis, which is again multipart, comprises, on the one hand, a shell-shaped body made from a flexibly set plastic, preferably from an addition cross-linked two-component silicone rubber compound, which is welded into a plastic film and additionally a second chamber which adjoins the first chamber and can be filled with a filler material. The volume and the shape of the breast prosthesis can be approximated as closely as possible to the natural breast of the wearer by corresponding filling of this second chamber.

In breast prostheses of the prior art, however, it has not been taken into account to date that the moist and warm contact area between the prosthesis and the skin forms an excellent medium for the formation of microorganisms such as fungi and above all bacteria. Cicatrized tissue can primarily, but not exclusively, become inflamed as a consequence. Allergic reactions or the formation of odor or spots due to decomposition of the perspiration are likewise unpleasant side-effects for the wearer. This problem has not yet been combated in the prior art.

This problem appears to be relevant, however. DE 101 07 189 A1 discloses adhesive patches for the securing of prostheses, inter alia breast prostheses. The components of the adhesive means used should be perfectly tolerated by the skin in accordance with the invention, in particular because the adhesive means remains in longer lasting contact with particularly sensitized skin. Antibacterial reagents are proposed as possible helpful additives, among many others, for the adhesive means. The patent specification U.S. Pat. No. 6,786,798 B1 discloses a brassiere for direct, post-operative use after various types of breast surgery. To reduce the post-operative risk of inflammation, it contains antimicrobially active fibers, for example such with the admixture of salts containing silver.

It is now the aim of the present invention to develop a breast prosthesis which maintains the favorable properties of the breast prostheses from the prior art and additionally acts antimicrobially at the contact points with the skin. The comfort in wear and the well-being of the woman should hereby be further improved and the formation of odor and spots, inflammation of the skin and, very generally, phenomena which can arise on the skin due to microorganisms can be largely suppressed.

This is achieved by a removable, external breast prosthesis, comprising a body substantially corresponding to the shape and firmness of the female breast and made from one or more flexible plastic parts, characterized in that the side of the prosthesis facing the skin has antimicrobial properties due to an antimicrobial coating and/or to an antimicrobial material in the matrix.

An embodiment of the invention includes a removable, external breast prosthesis, comprising a body substantially corresponding to the shape and firmness of the female breast and made from one or more flexible plastic parts, characterized in that the side of the prosthesis facing the skin has antimicrobial properties due to an antimicrobial coating and/or to an antimicrobial material in the matrix, and with the flexible plastic parts comprising a flexible plastic welded in a plastic envelope. In a preferred embodiment, this flexible plastic is an addition cross-linking two-component silicone rubber compound. The outer side and/or inner side of the plastic envelope can individually comprise suitable plastics which are preferably welded to one another along their peripheral margin by a peripheral weld seam. Plastic envelopes made of polyurethane are particularly preferred.

Further embodiments of the invention comprise breast prostheses of the prior art, characterized in that the side of the prosthesis facing the skin has antimicrobial properties due to an antimicrobial coating and/or to an antimicrobial material in the matrix.

A specific embodiment of the invention relates to breast prostheses which have a PCM (phase change material) material at their side facing the body which has a phase conversion temperature in the region of the body temperature, characterized in that the side of the prosthesis facing the skin has antimicrobial properties due to an antimicrobial coating and/or to an antimicrobial material in the matrix. This counters a possible heat build-up at the skin during wear.

Within the framework of the invention, further breast prostheses are conceivable whose side facing the skin has tixotropic properties and which achieve an ideal adaptation of the prosthesis to the uneven scar tissue or the uneven tissue after a partial amputation of the breast, characterized in that the side of the prosthesis facing the skin has antimicrobial properties due to an antimicrobial coating and/or to an antimicrobial material in the matrix. Alternatively or additionally to the tixotropic material, a corresponding adaptation can also take place via cross-linkable material which substantially maintains its shape after once being fit.

A further embodiment includes breast prostheses which have a fillable chamber to enable an individual volume and shape adaptation, characterized in that the side of the prosthesis facing the skin has antimicrobial properties due to an antimicrobial coating and/or to an antimicrobial material in the matrix.

The present invention includes all of the aforesaid embodiments either taken per se or with combined elements under the precondition that the side of the prosthesis facing the skin has antimicrobial properties due to an antimicrobial coating and/or to an antimicrobial material in the matrix.

The invention includes removable, external breast prostheses, with the side facing the skin having a film which has an antimicrobial coating and/or an antimicrobial material in the film matrix. Since such a film does not itself adhere to the skin, this embodiment of the breast prosthesis is either held in position via a brassiere or can, however, be combined with one of the following embodiments.

Preferred breast prostheses in accordance with the invention thus have an attachment fixedly or releasably on the side facing the skin. A preferred attachment comprises a textile which has an antimicrobial coating and/or an antimicrobial material in the textile matrix. This textile can be welded into a film or be disposed openly. The textile preferably comprises breathable fibers, which are compatible with the skin and have suitable properties with respect to the conducting away of perspiration and heat. Suitable fibers include, for example, fibers made of a thermoplastic material, cotton and microfibers made of nylon, spandex or other materials. It is furthermore conceivable to admix polyamide fibers or polyester fibers in a desired ratio which can be welded to the plastic body. The textile can preferably be adhesively connected to the skin. Suitable adhesive means are discussed in the following paragraph. A preferred manner to connect a textile adherable to the skin to the prosthesis is a hook and loop connection. It is likewise conceivable within the framework of the invention that a prosthesis containing textile is not adhered to the skin of the patient, but is only held in position via a brassiere.

In a further embodiment, the removable, external breast prosthesis in accordance with the invention has at its side facing the skin, fixedly or releasably, an adhesive material which has an antimicrobial coating and/or an antimicrobial material in the matrix. This adhesive material is preferably an adhesive silicone, but other suitable hydrophobic adhesives means are conceivable. Such adhesives means include inter alia two-block or multiblock elastomers, e.g. of the type A-B or A-B-A. Suitable elastomers include those with blocks of polystyrene (S), polyethene (E), polypropene (P) and polybutene (B), for example in the arrangement SEBS or SEPS. The use of polyurethane and acrylates parallel to EP 1 410 772 is conceivable. In addition to antimicrobial materials, resins can also be admixed to such a mixture of synthetic elastomers and to an adhesive silicone to increase adhesiveness or plasticizers can be admixed.

A further preferred embodiment of a breast prosthesis in accordance with the invention has an attachment (e.g. a textile) or a coating at the side facing the skin which contains biological material. Said attachment or coating can in turn have an antimicrobial coating and/or an antimicrobial material in the matrix. In a particularly preferred embodiment, the biological material acts antimicrobially by dispensing of antimicrobial substances itself. An example for this is formed by bioactive substances in bamboo leaves which include ingredients acting in an antimicrobial or antioxidative manner such as polyphenols, in particular flavonoids.

Suitable antimicrobial active ingredients include those with a specific effect, for example bactericides, fungicides or antiparasitics as well as substances or disinfectants acting non-specifically antimicrobially which can also kill spores and viruses in addition to bactericidal, fungicidal and antiparasitic effects.

All antibiotics and antifungal agents are, for example, covered by substances with a specific effect which are suitable for mixing into a coating or into a matrix. A mixing in of antibiotics from the class of the β lactams (optionally together with β lactamase inhibitors) is conceivable. The use of disinfectants is, however, preferred which act non-specifically against all kinds of microorganisms such as bacteria, spores, fungi, parasites and viruses. Those disinfectants are suitable among disinfectants which satisfy special demands corresponding to this special application with respect to the skin compatibility.

In a particularly preferred embodiment, the use of silver as the antimicrobial substance is provided in the present invention. The silver can be applied to the surface or mixed into a matrix in the form of ions or can be present in very finely distributed metallic form. It is worked into the matrix/material of a textile, of an adhesive silicone, of a plastic layer, of a biological material or of a film or is applied thereto as a coating as colloidal silver, as microsilver, as nanosilver, as silver threads or as a silver coating.

Possible coating processes such as are used by the present invention for the application of the antimicrobial substances to the side of the prosthesis facing the skin include chemical and physical gas phase deposition, mechanical application, injection and thermal injection, dip lacquering, spraying, evaporation coating or also electroplating in the case of elementary silver. The bonding of the antimicrobial material into the film matrix typically already takes place in its production phase, with the silver being able to be introduced into the solution in a casting film by mixing and homogenizing and with an extrusion film being able to be produced via a master batch or metering in the extruder.

The bonding of the antimicrobial material into the substance matrix typically already takes place in its production phase.

1. A removable, external breast prosthesis comprising a body substantially corresponding to the shape and firmness of the female breast and made from one or more flexible plastic parts, wherein
the side of the prosthesis facing the skin has antibacterial properties due to an antimicrobial coating and/or to an antimicrobial material in the matrix.

2. The removable, external breast prosthesis of claim 1, wherein the flexible plastic parts comprise an addition cross-linking two-component silicone rubber compound which are welded into a plastic envelope.

3. The removable, external breast prosthesis of claim 1, wherein the side of the prosthesis facing the skin has a film which has an antimicrobial coating and/or an antimicrobial material in the film matrix.

4. The removable, external breast prosthesis of claim 1, wherein the side of the prosthesis facing the skin has a fixed or releasable plastic layer which has an antimicrobial coating and/or an antimicrobial material in the plastic matrix.

5. The removable, external breast prosthesis of claim 1, wherein the side facing the skin is fixedly or releasably equipped with an adhesive material and/or with an attachment.

6. The removable, external breast prosthesis of claim 5, wherein the attachment has a textile which has an antimicrobial coating and/or an antimicrobial material in the textile matrix.

7. The removable, external breast prosthesis of claim 5, wherein the adhesive material includes an adhesive silicone which has an antimicrobial coating and/or an antimicrobial material in the silicone matrix.

8. The removable, external breast prosthesis of claim 5, wherein the adhesive material includes a hydrophobic elastomer which has an antimicrobial coating and/or an antimicrobial material in the matrix.

9. The removable, external breast prosthesis of claim 5, wherein the adhesive material and/or the attachment comprises a biological material.

10. The removable, external breast prosthesis of claim 9, wherein the biological material includes an antimicrobial coating and/or an antimicrobial material in the matrix.

11. The removable, external breast prosthesis of claim 9, wherein the biological material acts antimicrobially by dispensing of antimicrobial substances itself.

12. The removable, external breast prosthesis of claim 9, wherein the biological material originates from the bamboo plant.

13. The removable, external breast prosthesis of claim 1, wherein the antimicrobial material includes silver ions and/or metallic silver.

14. The removable, external breast prosthesis of claim 2, wherein the antimicrobial material includes silver ions and/or metallic silver.

15. The removable, external breast prosthesis of claim 3, wherein the antimicrobial material includes silver ions and/or metallic silver.

16. The removable, external breast prosthesis of claim 4, wherein the antimicrobial material includes silver ions and/or metallic silver.

17. The removable, external breast prosthesis of claim 5, wherein the antimicrobial material includes silver ions and/or metallic silver.

18. The removable, external breast prosthesis of claim 6, wherein the antimicrobial material includes silver ions and/or metallic silver.

19. The removable, external breast prosthesis of claim 7, wherein the antimicrobial material includes silver ions and/or metallic silver.

20. The removable, external breast prosthesis of claim 8, wherein the antimicrobial material includes silver ions and/or metallic silver.
